(19) United States
(12) Patent Application Publication
(10) Pub. No.: US 2012/0157777 A1
(43) Pub. Date: Jun. 21, 2012

(54) ADJUSTABLE HEIGHT MULTIPLE INSTRUMENT ACCESS SEAL ANCHOR MEMBER

(76) Inventor: Greg Okoniewski, North Haven, CT (US)

(21) Appl. No.: 13/224,354

(22) Filed: Sep. 2, 2011

Related U.S. Application Data
(60) Provisional application No. 61/424,765, filed on Dec. 20, 2010.

(51) Int. Cl. A61B 1/32

(52) U.S. Cl. 600/201

(57) ABSTRACT

A surgical apparatus for positioning within a tissue tract accessing an underlying body cavity includes a seal anchor member that includes a ring and a disc that are repositionable relative to one another, such that the height of the seal anchor member is adjustable and the angular positions of the disc and the ring, relative to one another, are adjustable.

### CROSS REFERENCE TO RELATED APPLICATION

The present application claims the benefit of and priority to U.S. Provisional Application Ser. No. 61/424,765 filed on Dec. 20, 2010, the entire contents of which are incorporated herein by reference.

### BACKGROUND

1. Technical Field

The present disclosure relates generally to a surgical apparatus for positioning within a tissue tract accessing an underlying body cavity for use in minimally invasive surgical procedures, such as endoscopic and/or laparoscopic procedures, and more particularly, relates to a seal anchor member providing multiple instrument access through a single incision.

2. Description of Related Art

Increasingly, many surgical procedures are performed through small incisions in the skin. As compared to the larger incisions typically required in traditional procedures, smaller incisions result in less trauma to the patient. By reducing the trauma to the patient, the time required for recovery is also reduced. Generally, the surgical procedures that are performed through small incisions in the skin are referred to as “endoscopic”. If the procedure is performed on the patient's abdomen, the procedure is referred to as “laparoscopic”. Throughout the present disclosure, the term “minimally invasive” is to be understood as encompassing both endoscopic and laparoscopic procedures.

During a typical minimally invasive procedure, surgical objects, such as surgical access devices (e.g., trocar and cannula assemblies) or endoscopes, are inserted into the patient's body through the incision in tissue. In general, prior to the introduction of the surgical object into the patient's body, insufflation gas is used to enlarge the area surrounding the target surgical site to create a larger, more accessible work area. Accordingly, the maintenance of a substantially fluid-tight seal is desirable so as to inhibit the escape of the insufflation gas and the deflation or collapse of the enlarged surgical site.

To this end, various access devices with sealing features are used during the course of minimally invasive procedures to provide an access for surgical objects to enter the patient's body. However, a continuing need exists for an access port, which can position the access port with relative ease and with minor inconvenience for the surgeon.

### SUMMARY

Disclosed herein is a surgical apparatus for positioning within a tissue tract for accessing an underlying body cavity. The surgical apparatus includes a seal anchor member having a longitudinal axis and including a disc and a ring. The disc and the ring are repositionable relative to the longitudinal axis and with respect to one another.

The disc is disposed at the distal or leading end of the seal anchor member. The disc may include one or more ports that are adapted to receive, in a substantially sealed relation, a surgical instrument, e.g., a cannula. The disc may be formed from a semi-rigid material to inhibit damage, e.g., bruising, to the surrounding tissue, and to facilitate a range of instrument motion inserted in the one or more ports of the disc.

The ring is adapted to provide rigidity to the seal anchor member and is disposed at or near the proximal or trailing end of the seal anchor member. The ring also facilitates anchoring the seal anchor member within a tissue tract. The ring and disc may be operatively coupled to one another by one or more positioning cables. By adjusting the length of the portions of the positioning cable that are positioned between the ring and the disc, the distance between the ring and the disc is adjustable. In addition, the angular position of the ring and the disc relative to each other and the longitudinal axis are similarly adjustable.

The positioning cable may be a drawstring that is affixed to the disc and passes through a lumen extending longitudinally through the disc. The interaction between the ring and the drawstring may be that of a ratchet-type mechanism, e.g., the drawstring is only translatable in one direction. This may be accomplished by positioning a plurality of fingers that are biased toward an expanded state and that interact with the lumen through which the drawstring is translated. The fingers facilitate maintaining the distance between the ring and the disc in the absence of a force applied upon the positioning cable. The fingers may be angled such that the fingers disposed on either side of the drawstring move toward one another when the drawstring is pulled in one direction and the fingers move away from one another when the drawstring is pulled in the opposite direction. Alternatively, or in addition to a ratchet-type mechanism, a clamp may secure the ring to the drawstring at a desired position.

Furthermore, the disc and the ring are configured and adapted to angularly translate relative to one another, thereby transitioning the seal anchor member between an expanded or first position, in which the disc and ring are substantially perpendicular to the longitudinal axis of the seal anchor member, and a collapsed or second position in which the disc and the ring are substantially co-axial or parallel with the longitudinal axis of the seal anchor member. By transitioning the seal anchor member to the second position, in which the disc and ring are substantially co-axial with the longitudinal axis of the seal anchor member, the width of the seal anchor member is reduced to facilitate introduction of the seal anchor member into an incision made in a tissue or into a naturally occurring orifice (e.g., anus or vagina). Once positioned within the incision, the seal anchor member may be transitioned to the first position in which the disc and ring are substantially perpendicular to the longitudinal axis of the seal anchor member.

The seal anchor member may include a bag construct to house the disc and the ring. The bag construct may have a double wall with the ring disposed between the inner and outer walls. In other embodiments, there may be only an outer wall. By drawing the positioning cables or drawstring through the lumens of the ring, the ring translates through the bag construct and the ring is repositioned with respect to the disc. When the seal anchor member in the expanded or first position, the walls of the bag construct are configured to provide some degree of rigidity to facilitate a sealed relation with the tissue tract in which the seal anchor member is placed.

These and other embodiments of the present disclosure will be described in greater detail below with reference to the appended figures.

### DESCRIPTION OF THE DRAWINGS

By way of description only, embodiments of the disclosure will be described with reference to the accompanying drawings, in which:

FIG. 1 is a perspective view of a surgical apparatus including a bag construct for positioning within a tissue tract accessing an underlying cavity according to the present disclosure shown in a first condition;

FIG. 1A is an enlarged view of the indicated area of FIG. 1 shown with the bag construct removed;

FIG. 2 is a perspective view of the surgical apparatus of FIG. 1 shown in a second condition;

FIG. 2A is a perspective view of the surgical apparatus of FIG. 1 shown in a third condition; and

FIG. 3 is a perspective view of the surgical apparatus of FIG. 1 shown positioned within tissue.

### DETAILED DESCRIPTION

Particular embodiments of the present disclosure will be described herein with reference to the accompanying drawings. As shown in the drawings and as described throughout the following descriptions, and is traditional when referring to relative positioning on an object, the term “proximal” refers to the end of the apparatus that is closer to the user and the term “distal” refers to the end of the apparatus that is farther from the user. In the following description, well-known functions or constructions are not described in detail to avoid obscuring the present disclosure in unnecessary detail. The “trailing” end of the surgical apparatus disclosed herein is at the “proximal” end of the apparatus. The “leading” end of the surgical apparatus is at the “distal” end of the apparatus.

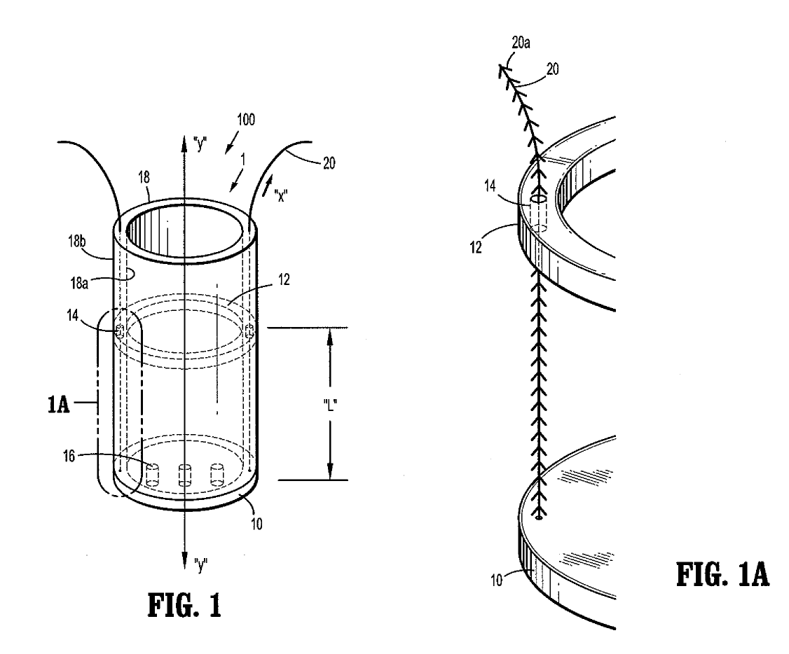

A surgical apparatus 100 for positioning within a tissue tract accessing an underlying body cavity will now be described with reference to FIGS. 1-3. The surgical apparatus 100 includes a seal anchor member 1. As shown best in FIG. 1, the seal anchor member 1 includes bag construct 18 housing a disc 10, at the distal or leading end of the seal anchor member, and a ring 12 positioned proximal to the disc 10. The bag construct 18 is secured to or affixed to the disc 10. The bag construct 18 may include an outer wall 18a and an inner wall 18b, between which the ring 12 may be positioned, as shown in FIG. 1. In some embodiments, however, the bag construct 18 may include a single walled surface extending longitudinally along the outer surface of the seal anchor member 1.

The disc 10 and the ring 12 may be operably coupled to one another by one or more positioning cables 20. Each positioning cable 20 may be secured to the disc 10 and be operably coupled to the ring 12. For example, the ring 12 may include one or more lumens 14, each lumen 14 being configured and adapted to receive a positioning cable 20 therethrough.

The positioning cable 20 may function as a drawstring, such that that pulling the positioning cable 20 through the lumen 14 in a proximal direction as indicated by directional arrow “X” (FIG. 1) will bring the disc 10 in closer proximity to the ring 12, reducing distance “L” between them. As shown in FIG. 1A, the positioning cable 20 may include a plurality of fingers 20a. The fingers 20a are transitionable between an expanded and a contracted position. In particular, the fingers 20a may be biased open and may be angled. The angled position of the fingers 20a permits translation of the positioning cable 20 through the lumen 14 of the ring 12, as indicated by directional arrow “X”, and inhibits translation of the positioning cable 20 through the lumen 14 in the opposite direction. In particular, application of a force to the positioning cable 20 in a direction opposite that indicated by directional arrow “X” will result in the further expansion of the fingers 20a and will inhibit translation of the positioning cable 20. In contrast, application of a force in the direction of directional arrow “X” will result in the fingers collapsing and will facilitate translation of the positioning cable 20 by reducing the frictional interaction between the positioning cable 20 and the lumen 14. Moreover, the amount of force required to overcome the frictional relationship between the fingers 20a and the lumen 14 is dependent upon the angle of the fingers 20a with respect to the longitudinal axis of the positioning cable 20. While FIG. 1A depicts the fingers 20a angled to facilitate translation of the disc 10 closer to the ring 12, the fingers 20a may be angled to facilitate translation of the positioning cable in the opposite direction. Alternatively, the fingers 20a may be substantially perpendicular to the longitudinal axis of the positioning cable 20 and may be biased open such that the force required for translation of the positioning cable 20 in either direction through the lumen 14 is the substantially equal.

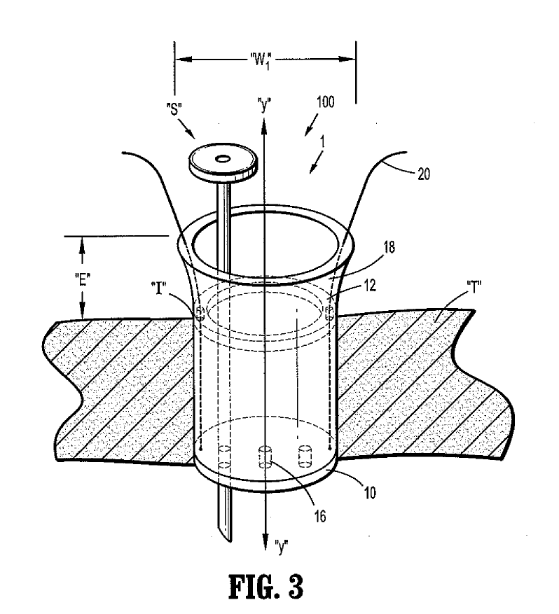

As discussed above, pulling the positioning cable 20 through lumen 14 will adjust the height “L” of the seal anchor member 1. In particular, the surgeon may adjust the height “L” by pulling on the positioning cable in the direction indicated by arrow “X”, while pressing on the ring 12 to ensure that the ring 12 does not move with the positioning cable 20, thereby pulling the positioning cable 20 through the lumen 14. In so doing, the length of cable between the ring 12 and the disc 10, which corresponds to the height “L” of the seal anchor member 1, will be lessened. The ring 12 may be translated through the bag construct 18 and the excess bag material “E” (FIG. 3), i.e., the portion of the bag construct that is proximal to the ring 12, may be trimmed or rolled.

The seal anchor member 1 is configured and adapted to be placed in an incision “I” of a tissue “T” in a substantially sealed relation therewith, as shown in FIG. 3. To facilitate placement of the seal anchor member 1 within an incision “I” of a tissue “T”, the position of the ring 12 and disc 10 with respect to each other and with respect to longitudinal axis “Y” may be adjusted. In particular, the orientation of the disc 10 and the ring 12 may be positioned at an angle with respect to longitudinal axis “Y”. By rotating the disc 10 and/or the ring 12 to a different angle with respect to the longitudinal axis “Y”, the insertion width of the seal anchor member 1 may be adjusted amongst a first width “W1”, a second width “W2”, and a third width “W3”. It is to be understood, that the seal anchor member 1 may be adjusted to have a plurality of widths including, but not limited to, a first width “W1”, a second width “W2”, and a third width “W3”. By rotating the disc 10 and the ring 12 to be parallel with the longitudinal axis “Y”, the width of the seal anchor member 1 becomes narrower, thereby facilitating placement of the seal anchor member 1 within the incision “I”.

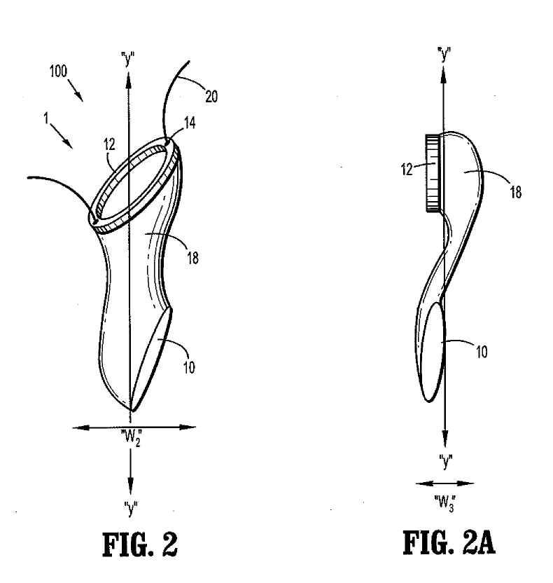

After placing the seal anchor member 1 in a collapsed state, as shown in FIGS. 2-2A, the ring 12 and the disc 10 may be rotated to be substantially perpendicular with the longitudinal axis “Y” such that the seal anchor member 1 is an expanded state. In the expanded state as shown in FIG. 3, the seal anchor member 1 conforms to the shape of the incision “I” such that the seal anchor member 1 maintains a substantially sealed relationship with the incision “I”. Once in the expanded state such that the ring 12 and disc 10 are substantially perpendicular to the longitudinal axis “Y” and/or such that the seal anchor member 1 conforms to the contours of the incision “I”, the height of the seal anchor member 1 may be adjusted by adjusted the distance between the ring 12 and the disc 10, as described above and by rolling or trimming the excess material “E” of the bag construct 18. In some procedures, such as those involving pliable or angled instrumentation, a surgeon may desire to angle the seal anchor member 1 by rotating the ring 12 to be at an angle with the longitudinal axis “Y”.

The disc 10 includes one or more ports 16. Each port 16 is adapted and configured to receive a surgical instrument “S”, e.g., a cannula, therein in a substantially sealed relationship. While the ring 12 is shown as having a single aperture, the ring 12 may in some embodiments have a disc-like configuration including one or more longitudinally extending openings. The disc 10 may be formed from a semi-rigid material to facilitate a range of motion of the surgical instrument “S” inserted within the ports 16 of the disc 10.

It will be understood by those skilled in the art that various modifications and changes in form and detail may be made therein without departing from the scope and spirit of the present disclosure. Accordingly, modifications and changes in form and detail may be made therein without departing from the scope and spirit of the present disclosure.

What is claimed is:

1. A surgical apparatus for positioning within a tissue tract accessing an underlying body cavity, comprising:
a seal anchor member having a longitudinal axis and including:
a disc including one or more ports that longitudinally extend through the disc; and
a ring, wherein the disc and the ring being are repositionable relative to the longitudinal axis and with respect to one another.

2. The surgical apparatus of claim 1, wherein the disc and the ring are distally and angularly translatable relative to one another.

3. The surgical apparatus of claim 1, wherein the disc is positioned distally relative to the ring.

4. The surgical apparatus of claim 1, wherein the one or more ports are adapted to receive a surgical instrument therein in a substantially sealed relation.

5. The surgical apparatus of claim 1, wherein the disc and the ring are angularly repositionable with respect to the longitudinal axis.

6. The surgical apparatus of claim 1, wherein the disc and the ring are configured and adapted to transition between a first position substantially perpendicular to the longitudinal axis and a second position substantially co-axial or parallel with the longitudinal axis.

7. The surgical apparatus of claim 1, wherein the disc and the ring are operably coupled to one another by one or more positioning cables.

8. The surgical apparatus of claim 7, wherein the one or more positioning cables are drawstrings.

9. The surgical apparatus of claim 8, wherein a drawstring is affixed to the disc.

10. The surgical apparatus of claim 9, wherein the ring includes a longitudinally extending lumen for receipt of the drawstring.

11. The surgical apparatus of claim 8, wherein the drawstring includes a plurality of fingers adapted interact with the lumen to permit translation of the drawstring through the lumen in only one direction.

12. The surgical apparatus of claim 1, further comprising a bag construct housing the disc and the ring therein.

13. The surgical apparatus of claim 1, wherein the disc is formed from a semi-rigid material.
